(19) United States
(12) Patent Application Publication
(10) Pub. No.: US 2011/0312977 A1
(43) Pub. Date: Dec. 22, 2011

(54) PROCESS FOR THE PREPARATION OF VORICONAZOLE

(75) Inventors: Francis Paul D'Souza, Navi Mumbai (IN); Premkumar Ramraj Yadav, Navi Mumbai (IN); Shankar Sanganabhatla, Navi Mumbai (IN); Mubeen Ahmed Khan, Navi Mumbai (IN)

(73) Assignee: GLENMARK GENERICS LIMITED, Mumbai (IN)

(21) Appl. No.: 13/148,522

(22) PCT Filed: Feb. 4, 2010
(86) PCT No.: PCT/IN2010/000065

§ 371 Date: Aug. 9, 2011

(30) Foreign Application Priority Data
Feb. 17, 2009 (IN) 342/MUM/2009

(51) Int. Cl. A61K 31/506; A61P 31/10; C07D 403/06

(52) U.S. Cl. 514/256; 544/333

(57) ABSTRACT

The present invention provides a process for preparation of racemic voriconazole in a single reaction vessel. The present invention also provides a process for preparation of voriconazole using racemic voriconazole and the process of making it therewith.

### PRIORITY

This application is a 35 U.S.C. 371 National Stage Filing of International Application No. PCT/IN2010/000065, filed Feb. 4, 2010, which claims priority under 35 U.S.C. 119 (a-d) to Indian Provisional Application No. 342/MUM/2009 filed on Feb. 17, 2009, entitled “PROCESS FOR THE PREPARATION OF VORICONAZOLE”, the contents of which are incorporated by reference herein.

### BACKGROUND OF THE INVENTION

1. Technical Field

The present invention generally relates to a process for the preparation of (2R,3S/2S,3R)-2-(2,4-difluorophenyl)-3-(5-fluoropyrimidin-4-yl)-1-(1H-1,2,4-triazol-1-yl)-2-butanol (racemic voriconazole) or a pharmaceutically acceptable salt thereof in a single reaction vessel, and a process for its conversion into voriconazole or a pharmaceutically acceptable salts thereof.

The present invention also provides crystalline racemic voriconazole Form A, processes for preparation thereof.

2. Description of the Related Art

Voriconazole, also known as (2R,3S)-2-(2,4-difluorophenyl)-3-(5-fluoropyrimidin-4-yl)-1-(1H-1,2,4-triazol-1-yl)-2-butanol, Formula (I).

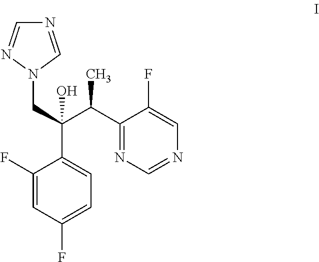

Voriconazole has an empirical formula of C₁₆H₁₄F₃N₅O and a molecular weight of 349.3. Voriconazole is a commercially marketed pharmaceutical substance known to be useful for the treatment of some fungal infections under the brand name VFEND® in the form of 200 mg per vial injectable formulation, 50 mg and 200 mg solid oral tablet formulations and an oral suspension containing 200 mg of voriconazole/5 ml.

U.S. Pat. No. 5,567,817 (the '817 patent) discloses triazole antifungal agents such as voriconazole, or a pharmaceutically acceptable salt thereof. The process disclosed in the '817 patent is schematically represented as follows:

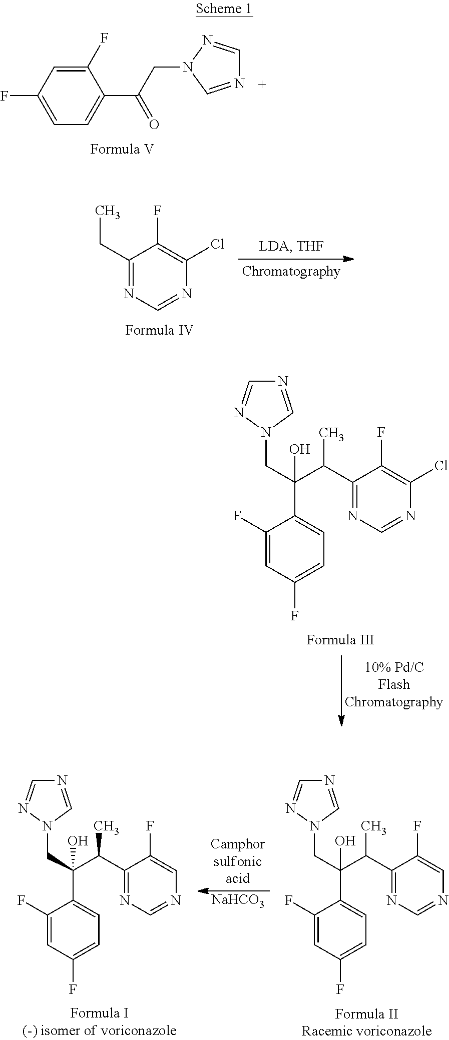

Scheme 1 of the '817 patent shows the preparation of voriconazole by the reaction of 1-(2,4-difluorophenyl)-2-(1H-1,2,4-triazole-1-yl)ethanone (Formula V) with 4-chloro-6-ethyl-5-fluoropyrimidine (Formula IV), in the presence of lithium diisopropylamide (LDA) and in a tetrahydrofuran (THF) medium, to yield 3-(4-chloro-5-fluoropyrimidin-6-yl)-2-(2,4-difluorophenyl)-1-(1H-1,2,4-triazole-1-yl)butan-2-ol (Formula III) as a residue. Column chromatography of the residue on silica gave 2R, 3S and 2S, 3R enantiomeric pair of 3-(4-chloro-5-fluoropyrimidin-6-yl)-2-(2,4-difluorophenyl)-1-(1H-1,2,4-triazole-1-yl)butan-2-ol (Formula III), which is further dehalogenated with 10% Pd/C catalyst in ethanol, in the presence of sodium acetate, followed by flash chromatographic separation yields (2R,3 S/2S,3R)-2-(2,4-difluorophenyl)-3-(5-fluoropyrimidin-4-yl)-1-(1H-1,2,4-triazol-1yl) butan-2-ol (racemic voriconazole) (Formula II). The racemic voriconazole compound of Formula II on resolution with R-(−)-10-camphor sulfonic acid in methanol gives a corresponding camphorsulfonate salt, which on hydrolysis with aqueous sodium bicarbonate solution gives voriconazole (Formula I).

Patent Publication WO 2006/065726 (the '726 patent publication) discloses a process for the preparation of voriconazole by the reaction of 1-(2,4-difluorophenyl)-2-(1H-1,2,4-triazole-1-yl)ethanone of Formula V with 4-chloro-6-ethyl-5-fluoropyrimidine of Formula IV, in the presence of lithium diisopropylamide. The resultant compound 3-(4-chloro-5-fluoropyrimidin-6-yl)-2-(2,4-difluorophenyl)-1-(1H-1,2,4-triazole-1-yl) butan-2-ol (Formula III) is isolated by crystallization from n-heptane at temperature between −10° C. to −15° C. as a crystalline solid, which is further dehalogenated with Raney nickel to provide racemic voriconazole (Formula II) followed by resolution with R-(−)-10-camphor sulfonic acid and subsequent hydrolysis with aqueous sodium bicarbonate solution gives voriconazole.

Patent Publication WO 2007/132354 (the '354 patent publication) discloses a process for the preparation of voriconazole by dehalogenation of hydrochloride salt of 3-(4-chloro-5-fluoropyrimidin-6-yl)-2-(2,4-difluorophenyl)-1-(1H-1,2,4-triazole-1-yl)butan-2-ol (Formula III) to form racemic voriconazole, followed by resolution with R-(−)-10-camphor sulfonic acid gives voriconazole camphor sulfonate salt as crystalline solid form-B, which upon hydrolysis with sodium bicarbonate gives voriconazole.

Patent Publication WO 2007/013096 (the '096 patent publication) discloses a process for the preparation of voriconazole by reaction of 1-(2,4-difluorophenyl)-2-(1H-1,2,4-triazole-1-yl)ethanone of Formula V with 4-chloro-6-ethyl-5-fluoropyrimidine of Formula IV, in the presence of lithium diisopropylamide to form 3-(4-chloro-5-fluoropyrimidin-6-yl)-2-(2,4-difluorophenyl)-1-(1H-1,2,4-triazole-1-yl)butan-2-ol as a crystalline solid by solvent crystallization at temperature between −10° C. to −15° C., which is further dehalogenated with Raney nickel, followed by resolution with R-(−)-10-camphor sulfonic acid and subsequent hydrolysis with aqueous sodium bicarbonate solution gives voriconazole.

The synthesis of voriconazole as discussed in the '817 patent involves the use of chromatographic separation of a 2R,35 and 2S,3R enantiomeric pair from the residue containing four possible enantiomers of the 3-(4-chloro-5-fluoropyrimidin-6-yl)-2-(2,4-difluorophenyl)-1-(1H-1,2,4-triazole-1-yl)butan-2-ol (Formula III), leading to poor yields. The process also involves chromatographic purification of racemic voriconazole (Formula II), resulting in a process that is expensive and difficult to operate on an industrial scale.

The '726 and '096 patent publications disclose the use of a solvent crystallization technique at low temperatures, about −15° C., in the isolation of 3-(4-chloro-5-fluoropyrimidin-6-yl)-2-(2,4-difluorophenyl)-1-(1H-1,2,4-triazole-1-yl)butan-2-ol (Formula III). This involves an additional manufacturing step and a decrease in the product yield.

The '354 patent publication discloses the use of 3-(4-chloro-5-fluoropyrimidin-6-yl)-2-(2,4-difluorophenyl)-1-(1H-1,2,4-triazole-1-yl)butan-2-ol (Formula III) as hydrochloric acid addition salt. This entails supplemental manufacturing steps of acid addition salt preparation and neutralization of the acid addition salt, subsequently leading to an increase in the manufacturing cycle time and a decrease in the product yield.

Generally, racemic voriconazole (Formula II) is highly soluble in polar solvents, which leads to incomplete precipitation of the product from reaction solution in which the product precipitated therefrom. In the process described in the '726 patent publication, the racemic voriconazole (Formula II) is precipitated from a highly polar solvent water; and similarly, the racemic voriconazole (Formula II) is precipitated from methanol in the '354 patent publication.

It would be desirable to provide a process for the preparation of racemic voriconazole, or a pharmaceutically acceptable salt thereof in a single reaction vessel, which is simple and cost effective; and a process for its use thereof in the preparation of voriconazole or a pharmaceutically acceptable salt thereof in a convenient, cost efficient manner and a commercial scale.

### SUMMARY OF THE INVENTION

The invention encompasses a process for the preparation of (2R,3S/2S,3R)-2-(2,4-difluorophenyl)-3-(5-fluoropyrimidin-4-yl)-1-(1H-1,2,4-triazol-1-yl)-2-butanol of Formula II (hereinafter referred to as “racemic voriconazole”) or a pharmaceutically acceptable salt thereof and its conversion into (2R,3S)-2-(2,4-difluorophenyl)-3-(5-fluoropyrimidin-4-yl)-1-(1H-1,2,4-triazol-1-yl)-2-butanol of Formula I (hereinafter referred to as “voriconazole”) or a pharmaceutically acceptable salt thereof with high product yield and quality. In particular, the invention encompasses a process for the preparation of racemic voriconazole in a single reaction vessel, which avoids filtration and drying problems, thereby increasing the yield, and decreasing the manufacturing cost.

The present invention provides a process for the preparation of 2-(2,4-difluorophenyl)-3-(5-fluoropyrimidin-4-yl)-1-(1-1H-1,2,4-triazol-1-yl)-2-butanol (racemic voriconazole) of formula II or a pharmaceutically acceptable salt thereof, comprising;
a) condensing 4-chloro-6-ethyl-5-fluoro pyrimidine of formula IV with 1-(2,4-difluorophenyl)-2-(1H-1,2,4-triazole-1-yl)ethanone of formula V in presence of an organic metallic base and an organic solvent S1,
b) concentrating the reaction mixture to obtain 3-(4-chloro-5-fluoropyrimidin-6-yl)-2-(2,4-difluorophenyl)-1-(1H-1,2,4-triazole-1-yl)butan-2-ol of formula III as residue,
c) dechlorination of the resulting compound of formula III as a residue under catalytic hydrogenation by using a metal catalyst in an organic solvent S2 to form a compound of formula II, and
d) isolating the racemic voriconazole of formula II or a pharmaceutically acceptable salt thereof.

The present invention provides a process for the preparation of racemic voriconazole, comprising forming a compound 3-(4-chloro-5-fluoropyrimidin-6-yl)-2-(2,4-difluorophenyl)-1-(1H-1,2,4-triazole-1-yl)butan-2-ol (Formula III), comprising the reactions a) and b) of the process, previously discussed above; wherein which the ensuing product is subsequently dechlorinated to yield racemic voriconazole, wherein said compound of Formula III, is not crystallized out before dechlorination.

The present invention provides a process for purification of racemic voriconazole, comprising the steps of
a) dissolving racemic voriconazole in a suitable organic solvent,
b) heating the mixture of a) at a temperature sufficient to obtain a solution,
c) adding an antisolvent to the resultant solution of b) to precipitate the racemic voriconazole or a pharmaceutically acceptable salt thereof,
d) cooling the precipitate and recovering the precipitate.

The present invention provides a process for purification of racemic voriconazole, comprising the steps of
a) dissolving racemic voriconazole in a suitable organic solvent, wherein the suitable organic solvent is selected from methanol, ethanol, isopropanol, butanol and mixtures thereof,
b) heating the mixture of a) at a temperature sufficient to obtain a solution,
c) adding an antisolvent to the resultant solution of b) to precipitate the racemic voriconazole or a pharmaceutically acceptable salt thereof, wherein the antisolvent is hydrocarbon solvents selected from n-hexane, n-heptane, pentane, cyclohexane and toluene and mixtures thereof,
d) cooling the precipitate and recovering the precipitate.

The present invention provides a crystalline form of racemic voriconazole (herein designated “Form A”).

The present invention provides a crystalline racemic voriconazole Form A, wherein the crystalline racemic voriconazole Form A has an XRD pattern substantially in accordance with FIG. 1.

The present invention provides a crystalline racemic voriconazole Form A, wherein the crystalline racemic voriconazole Form A has DSC thermogram substantially in accordance with FIG. 2.

The present invention provides a process for preparation of voriconazole; the process comprising converting a compound of racemic voriconazole or racemic voriconazole Form A prepared by the processes of the present invention to voriconazole.

The present invention provides a process for preparation of voriconazole comprising;
a) reacting the racemic voriconazole prepared by the process, herein described, with (1R)-(−)-10-camphorsulfonic acid,
b) neutralizing the resulting voriconazole (1R)-(−)-10-camphorsulfonate salt.

The present invention provides voriconazole substantially free of (2S,3R)-2-(2,4-difluorophenyl)-3-(5-fluoropyrimidin-4-yl)-1-(1,2,4-triazol-1-yl)-2-butanol of formula IA.

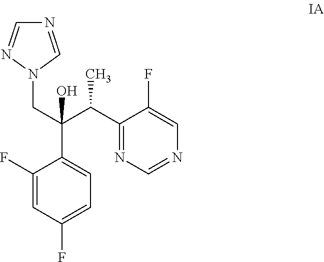

The present invention provides voriconazole substantially free of (2S,3S)-2-(2,4-difluorophenyl)-3-(5-fluoropyrimidin-4-yl)-1-(1,2,4-triazol-1-yl)-2-butanol of formula IB.

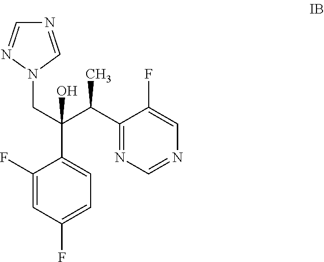

The present invention provides voriconazole substantially free of (2R,3R)-2-(2,4-difluorophenyl)-3-(5-fluoropyrimidin-4-yl)-1-(1,2,4-triazol-1-yl)-2-butanol of formula IC.

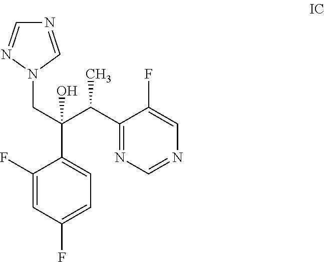

The present invention provides a pharmaceutical composition comprising a therapeutically effective amount of a voriconazole prepared by the processes of the present invention and at least one pharmaceutically acceptable carrier.

### DESCRIPTION OF THE DRAWINGS

FIG. 1 is a characteristic powder X-ray diffraction (PXRD) pattern of racemic voriconazole prepared according to Example 4.

FIG. 2 is a characteristic DSC thermogram of racemic voriconazole prepared according to Example 4.

### DETAILED DESCRIPTION OF THE INVENTION

The present invention provides an improved process for the preparation of voriconazole or a pharmaceutically acceptable salt thereof. In particular, the present invention provides a process to prepare voriconazole or a pharmaceutically acceptable salt thereof by a process for preparing racemic voriconazole or a pharmaceutically acceptable salt thereof in a single reaction vessel, which avoids filtration and drying steps, thereby increasing the yield, and decreasing the manufacturing cost.

Surprisingly, it has been found according to the present invention that racemic voriconazole or a pharmaceutically acceptable salt thereof can be synthesized in one step from intermediates 1-(2,4-difluorophenyl)-2-(1H-1,2,4-triazole-1-yl)ethanone (Formula V) and 4-chloro-6-ethyl-5-fluoropyrimidine (Formula IV) without crystallizing the resulting 3-(4-chloro-5-fluoropyrimidin-6-yl)-2-(2,4-difluorophenyl)-1-(1H-1,2,4-triazole-1-yl)butan-2-ol (Formula III) before the dechlorination reaction.

The present invention provides a process for preparation of racemic voriconazole or a pharmaceutically acceptable salt thereof in a single reaction vessel, comprising;
a) condensing 4-chloro-6-ethyl-5-fluoro pyrimidine (Formula IV) with 1-(2,4-difluorophenyl)-2-(1H-1,2,4-triazole-1-yl)ethanone (Formula V) in the presence of an organic metallic base and an organic solvent S1,
b) concentrating the reaction mixture to obtain 3-(4-chloro-5-fluoropyrimidin-6-yl)-2-(2,4-difluorophenyl)-1-(1H-1,2,4-triazole-1-yl)butan-2-ol (Formula III) as residue,
c) dechlorinating the resulting compound of b) above as a residue under catalytic hydrogenation by using a metal catalyst in an organic solvent S2 to form a compound of formula II, and
d) isolating the racemic voriconazole (Formula II) or a pharmaceutically acceptable salt thereof.

The starting material of the compound of formula IV may either be known compounds (illustratively, incorporated herein in its entirety, D. L. Comins et al, Heterocycles, 22, 339 (1984)) or compounds prepared by conventional procedures in accordance with literature precedents. The starting material of the compound of formula V may either be known compounds (illustratively, incorporated herein in their entirety, EP-A-44605, EP-A-69442 or GB-A-1464224) or may be prepared by similar methods to those described therefor.

Condensation of 1-(2,4-difluoro phenyl)-2-(1H-1,2,4-triazole-1-yl)ethanone (Formula V) and 4-chloro-6-ethyl-5-fluoro pyrimidine (Formula IV) is carried out in the presence of an organic metallic base and an organic solvent S1, to give 3-(4-chloro-5-fluoropyrimidin-6-yl)-2-(2,4-difluorophenyl)-1-(1H-1,2,4-triazole-1-yl)butan-2-ol (Formula III).

A suitable organic metallic base include, but are not limited to lithium diisopropylamide (complex form of n-butyl lithium and diisopropylamine, herein designated “LDA”), magnesium isopropylamide, sodium bis(trimethylsilyl)amide, potassium bis(trimethylsilyl)amide, butyl magnesium, zinc isopropyl amide, butyl zinc, lithium hexamethyl disilazane, sodium hexamethyl disilazane, magnesium hexamethyl disilazane. Preferably, LDA.

A suitable organic solvent S1 include, but are not limited to, ethers such as diethyl ether, dimethyl ether, diisopropyl ether, methyl tertiary-butyl ether, tetrahydrofuran, 1,4-dioxane and the like; aliphatic hydrocarbons such as C₁-C₁₀ straight chain or branched hydrocarbons such as n-hexane, n-heptane, cyclohexane, pentane and the like; and aromatic hydrocarbons such as toluene, xylene and the like; and mixtures thereof. Preferably, diisopropyl ether, n-hexane, n-heptane, tetrahydrofuran and mixtures thereof; more preferably mixture of tetrahydrofuran and hexane.

After the completion of the condensation reaction, the reaction mass can be quenched by adding a suitable acid such as acetic acid, hydrochloric acid and the like and then diluting with water. After removal of the solid that is produced, such as by filtration, the organic layer of the filtrate can be separated and washed with water. The resultant organic layer containing 3-(4-chloro-5-fluoropyrimidin-6-yl)-2-(2,4-difluorophenyl)-1-(1H-1,2,4-triazole-1-yl)butan-2-ol (Formula III) can be further processed directly in the same reaction vessel to form a compound of Formula II. Alternatively, the solvent from the organic layer may be removed by methods known in the art, for example evaporation at atmospheric pressure, evaporation under vacuum to form and isolate the 3-(4-chloro-5-fluoropyrimidin-6-yl)-2-(2,4-difluorophenyl)-1-(1H-1,2,4-triazole-1-yl)butan-2-ol (Formula III) as residue, preferably the method used for solvent removal is evaporation under vacuum. Neither chromatographic separation nor crystallization techniques are implemented to recover the product of Formula III, before it is subjected to further processing.

The '817 patent discloses isolation of 3-(4-chloro-5-fluoropyrimidin-6-yl)-2-(2,4-difluorophenyl)-1-(1H-1,2,4-triazole-1-yl)butan-2-ol (Formula III) by chromatographic separation. The '726 and '096 patent publications disclose solvent crystallization at relatively low temperatures as well as further processing steps such as drying the product to recover the compound of Formula III.

In contrast, the process herein described, for the preparation of the compound of Formula III avoids extraneous chromatographic separation and crystallization techniques for product recovery found in the art. The process herein described, for the preparation of the compound of Formula III, has the dual benefit of less manufacturing cycle time and improved product yield.

The present invention provides dechlorination of 3-(4-chloro-5-fluoropyrimidin-6-yl)-2-(2,4-difluorophenyl)-1-(1H-1,2,4-triazole-1-yl)butan-2-ol (Formula III), which is carried out under catalytic hydrogenation, in the presence of a metal catalyst in an organic solvent S2 optionally in the presence of a suitable base, to obtain racemic voriconazole optionally in the form of pharmaceutically acceptable salt thereof. Preferably, in the form of hydrochloride salt or racemic camphor sulfonic acid salt.

Suitable metal catalysts include, but are not limited to, palladium in the form of palladium on carbon or palladium salts such as palladium hydroxide, palladium hydroxide on carbon, and the like, Raney nickel, platinum, iridium, ruthenium, and the like; preferably, a palladium catalyst, or Raney nickel and most preferably, the metal catalyst is palladium on carbon. The palladium content in the catalyst may be about 2.5% to about 20% wt/wt % on carbon, preferably about 5% to about 15 wt/wt %, more preferably about 10 wt/wt %.

The suitable base includes, but is not limited to, sodium acetate, potassium acetate, ammonium formate and the like, and a mixture thereof, preferably the suitable base is sodium acetate.

The suitable organic solvent S2 includes, but is not limited to, alcohols such as C₁-C₄ straight chain or branched alcohols such as methanol, ethanol, isopropanol, n-propanol, butanol, isobutanol and the like, and mixtures thereof; esters such as methyl acetate, ethyl acetate, isopropyl acetate, tertiary butyl acetate and the like; and mixtures thereof; water and mixtures thereof; preferably the organic solvent S2 is ethyl acetate, methanol, ethanol, water, and mixtures thereof; more preferably the organic solvent S2 is ethyl acetate, water, and mixtures thereof.

The pressure for the dechlorination reaction can range from about 2 kg/cm²g to about 10 kg/cm²g by using hydrogen gas. Preferably at about 5 kg/cm²g to about 8 kg/cm²g, more preferably at about 5 kg/cm²g.

The temperature for dechlorination can range from about 20° C. to about 80° C. Preferably, at about 25° C. to about 35° C. The hydrogenation process may take from about 3 hours to about 20 hours. Preferably about 6 hours to about 14 hours depending upon the catalyst, pressure and temperature chosen.

The metal catalysts may be separated or recovered from the reaction mixture at the end of the hydrogenation reaction by any method known in the art. The reaction solution containing resultant target product may be diluted with an organic solvent and a base, wherein the organic solvent may be selected from esters such as methyl acetate, ethyl acetate, isopropyl acetate, tertiary butyl acetate and the like; halogenated solvents such as dichloromethane, dichloroethane, chloroform, carbon tetrachloride and the like, hydrocarbons such as n-hexane, n-heptane, cyclohexane, benzene, toluene and the like and mixtures thereof; water and mixtures thereof; preferably the solvent is ethyl acetate, dichloromethane, chloroform, toluene, water and mixtures thereof; more preferably ethyl acetate.

The base used can be any of those that are known in the art, for example sodium hydroxide, potassium hydroxide and the like, sodium carbonate, potassium carbonate and the like. Preferably the base is sodium carbonate.

The resultant target product is separated from the aqueous solution and extracted with an organic solvent, wherein the organic solvent may be selected from esters such as methyl acetate, ethyl acetate, isopropyl acetate, tertiary butyl acetate and the like; halogenated solvents such as dichloromethane, dichloroethane, chloroform, carbon tetrachloride and the like, hydrocarbons such as n-hexane, n-heptane, cyclohexane, benzene, toluene and the like and mixtures thereof. Preferably the solvent is ethyl acetate, dichloromethane, chloroform, toluene, and mixtures thereof, more preferably ethyl acetate.

The resultant organic solvent containing the target product, which is racemic voriconazole, is separated and may be concentrated under vacuum to get the residue by any method known in the art, for example distillation, evaporation, rotational drying (such as with the Buchi Rotavapor), freeze-drying, fluidized bed drying, flash drying, spin flash drying, and the like, preferably the separation technique is distillation under vacuum. The resultant racemic voriconazole may be isolated as its free base or its pharmaceutically acceptable salt form.

Racemic voriconazole obtained by the process described above may be dissolved in a suitable solvent. Suitable solvent include but are not limited to, C₁-C₄ alcohols such as methanol, ethanol, n-propanol, isopropanol, n-butanol, isobutyl alcohol, tertiary butyl alcohol and the like and mixtures thereof; esters such as ethyl acetate and the like, ketones such as acetone, ethyl methyl ketone and the like; and their mixtures, preferably the solvent is methanol, isopropanol, acetone, ethyl acetate or their mixtures.

Suitable acids used for formation of pharmaceutically acceptable salt form of racemic voriconazole, include but are not limited to inorganic acids such as hydrochloric acid, hydrobromic acid, sulphuric acid, phosphoric acid, nitric acid, and the like, organic acids such as acetic acid, citric acid, tartaric acid, camphorsulphonic acid and the like; preferably, hydrochloric acid and camphorsulphonic acid. Where the acid may be in the form of an aqueous, anhydrous or gas form, for example aqueous acid or solvent containing acid or gas containing acid, preferably a solvent containing acid can be used.

The temperature for dissolution of racemic voriconazole ordinarily range from about 0° C. to about 80° C. and preferably about 30° C. to about 75° C. or reflux temperatures of the solvents used. The suitable acid is added to the solution containing racemic voriconazole at a temperature ranging from about 20° C. to about 75° C. and preferably about 30° C. to about 50° C. The resulted mixture is stirred for about 10 minutes to about 24 hours, preferably about 30 minutes to about 10 hours at a temperature ranging from about 0° C. to about 75° C. and preferably about 20° C. to about 50° C. The racemic voriconazole salt precipitant is filtered. The resultant product may optionally be further dried.

Racemic voriconazole in pharmaceutically acceptable salt form obtained by the process described above may be neutralized to get racemic voriconazole as free base. The step of neutralization involves treating the pharmaceutically acceptable salt form of racemic voriconazole in a mixture of solvents, such as water and hydrocarbon solvents; hydrocarbon solvents such as dichloromethane, dichloroethane, toluene, preferably dichloromethane and water, and in the presence of a base such as sodium hydroxide, potassium hydroxide, sodium bicarbonate, potassium carbonate, sodium bicarbonate, and potassium bicarbonate and the like, preferably potassium carbonate. The layers obtained are separated; the organic layer is washed with water. The solvent is removed by distillation under vacuum at temperatures below about 70° C. Preferably, at below about 45° C.

The present invention provides advantageous processes for preparing racemic voriconazole. For instance, the process of the instant invention described herein, circumvents the step of crystallization of a key intermediate 3-(4-chloro-5-fluoropyrimidin-6-yl)-2-(2,4-difluorophenyl)-1-(1H-1,2,4-triazole-1-yl)butan-2-ol (Formula III). The processes of the present invention eliminate the chromatographic separation process of potential enantiomeric pairs.

The present invention provides a process for purification of racemic voriconazole, comprising:
a) dissolving racemic voriconazole in a suitable organic solvent,
b) heating the mixture of a) at a temperature sufficient to obtain a solution,
c) adding an antisolvent to the resultant solution of b) to precipitate the racemic voriconazole or a pharmaceutically acceptable salt thereof,
d) cooling the precipitate and recovering the precipitate.

In a) of the foregoing process, the racemic voriconazole free base obtained as described above may be dissolved in a suitable organic solvent. Suitable organic solvents include, but are not limited to alcohols such as C₁₋₄ alcohols selected from methanol, ethanol, isopropanol, n-propanol, butanol, isobutanol and the like; esters such as methyl acetate, ethyl acetate, isopropyl acetate, tertiary butyl acetate and the like; and halogenated solvents such as dichloromethane, dichloroethane, chloroform, carbon tetrachloride and the like; water and their mixtures, preferably the solvent used for dissolution is methanol, ethanol, isopropanol, ethyl acetate, water and their mixtures; more preferably the solvent used for dissolution is isopropanol, water or their mixtures. The volume of the solvent used to solubilize racemic voriconazole may range from about 0.2 volumes to about 5 volumes to the weight of the racemic voriconazole.

Then, the solution may be heated at a temperature of at least about 30° C. to about solvent reflux. Preferably, the solution is heated at about 30° C. to about 85° C., and more preferably at about 60° C. to about 70° C.

In c) of the foregoing process, a suitable antisolvent is added to the solution to precipitate the racemic voriconazole. Useful antisolvents include, but are not limited to, hydrocarbon solvents such as n-pentane, n-hexane, n-heptane, cyclohexane and the like; ethers such as dimethyl ether, diethyl ether, diisopropyl ether and the like; toluene and the like. Mixtures of any of these antisolvents are also contemplated. Preferably the antisolvent is n-heptane, n-hexane and mixtures thereof. The volume of antisolvent used to precipitate the solid can advantageously range from about 2 volumes to about 20 volumes with reference to volume of the solvent used for solubilizing racemic voriconazole. Generally, the antisolvent is added to the solution at a temperature ranging from about 20° C. to about 80° C. Preferably, at a temperature ranging from about 30° C. about 70° C., more preferably at about 50° C. to 60° C.

The racemic voriconazole can be recovered by any conventional technique known in the art, for example filtration. Typically, if stirring is involved, the temperature during stirring can range from about −10° C. to about 30° C., preferably at about 20° C. to about 25° C., more preferably at about 0° C. to 10° C. The resultant product may optionally be further dried. Suitably, drying can be carried out in a tray dryer, vacuum oven, air oven, fluidized bed drier, spin flash dryer, flash dryer and the like. The drying can be carried out at a temperature ranging from about 30° C. to about 90° C. The drying can be carried out for any desired time until the required product purity is achieved, e.g., a time period ranging from about 1 hour to about 20 hours, preferably about 10 hours.

The racemic voriconazole recovered using the purification process of the present invention is in crystalline Form A.

The present invention provides a racemic voriconazole in crystalline Form A.

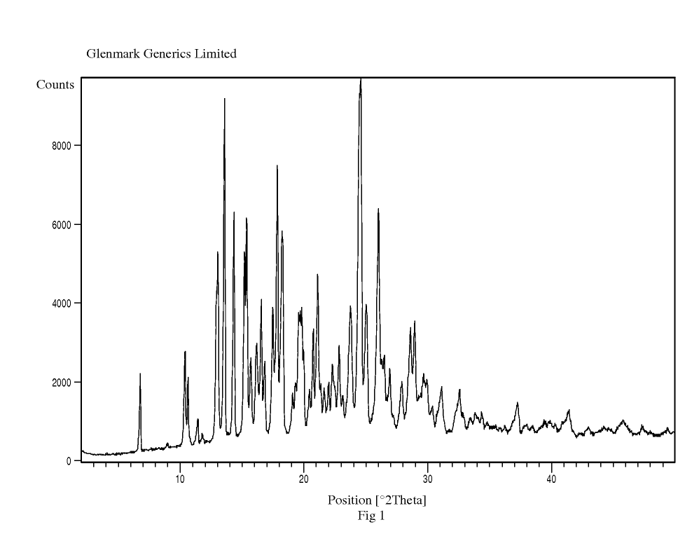

The present invention provides characterization via X-ray powder diffraction pattern and/or melting point of a racemic voriconazole in a crystalline form A, which is substantially in accordance with FIG. 1. The X-Ray powder diffraction can be measured by an X-ray powder Diffractometer equipped with a Cu-anode (λ=1.54 Angstrom), X-ray source operated at 45 kV, 40 mA and a Ni filter is used to strip K-beta radiation. Two-theta calibration is performed using an NIST SRM 640c Si standard. The sample was analyzed using the following instrument parameters: measuring range=2-50° 2θ; step width=0.017°; and measuring time per step=5 sec.

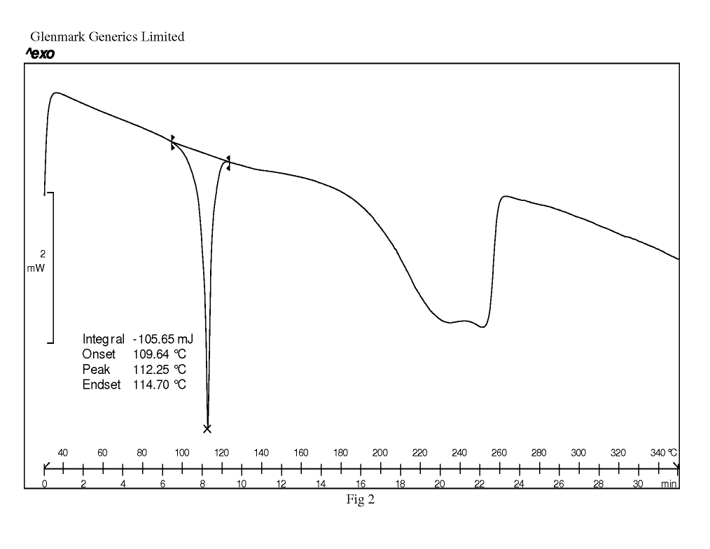

The present invention further provides a racemic voriconazole in crystalline Form A, with a differential scanning calorimetric thermogram, which is as substantially in accordance with FIG. 2. The scan is performed with a Differential Scanning calorimeter (DSC 822, Mettler Toledo) at a scan rate of 10° C. per minute with an Indium standard. Racemic voriconazole in crystalline Form A exhibits a predominant endotherm peak at about 112.25° C. Whereupon, the endotherm measured by a particular differential scanning calorimeter is dependent upon a number of factors, including the rate of heating (i.e., scan rate), the calibration standard utilized, instrument calibration, relative humidity, and upon the chemical purity of the sample being tested. Thus, an endotherm as measured by DSC on the instrument identified above may vary by as much as ±1° C. or even ±2° C.

The present invention provides a process for preparation of voriconazole, comprising providing a racemic voriconazole as obtained by the process described herein above, as a starting material or as an intermediate.

The present invention further provides a process for a preparation of a voriconazole, comprising;
a) reacting the racemic voriconazole obtained by the process defined herein above, with a (1R)-(−)-10-camphorsulfonic acid,
b) neutralizing the resulting voriconazole (1R)-(−)-10-camphorsulfonate salt.

The reaction of racemic voriconazole with (1R)-(−)-10-camphorsulfonic acid occurs in the presence of a suitable solvent to form the diastereomeric salt voriconazole (1R)-(−)-10-camphorsulfonate salt at ambient temperature. The suitable solvents include, but are not limited to, C₁-C₄ alcohols such as methanol, ethanol, n-propanol, isopropanol, n-butanol, isobutyl alcohol, tertiary butyl alcohol and the like and mixtures thereof; ketones such as acetone, ethyl methyl ketone and the like; or their mixtures, preferably the solvent is methanol, acetone, or their mixtures.

The neutralization step involves conversion of voriconazole (1R)-(−)-10-camphor sulfonate salt to voriconazole by the hydrolysis of the voriconazole (1R)-(−)-10-camphor sulfonate salt in a mixture of solvents, such as water and hydrocarbon solvents; hydrocarbon solvents such as dichloromethane, dichlorethane, toluene, preferably dichloromethane and water, and in the presence of a base such as sodium hydroxide, potassium hydroxide, sodium bicarbonate, potassium carbonate, sodium bicarbonate, and potassium bicarbonate and the like, preferably sodium hydroxide. The layers obtained are separated; the organic layer is washed with water. The solvent is removed by distillation under vacuum at temperatures below about 70° C. Preferably, at below about 45° C. The obtained crude voriconazole is crystallized from suitable organic solvents to get pure voriconazole.

Suitable organic solvents for crystallization of voriconazole are selected from, but not limited to, water; C₁-C₄ alcohols such as methanol, ethanol, n-propanol, isopropanol, n-butanol, isobutyl alcohol, tertiary butyl alcohol and the like and mixtures thereof; esters such as methyl acetate, ethyl acetate, isopropyl acetate, tertiary butyl acetate and the like; ketones such as acetone, ethyl methyl ketone, and the like and mixtures thereof; hydrocarbons such as n-hexane, n-heptane, cyclohexane, toluene, xylene and the like and mixtures thereof. Preferably the solvent used for dissolution is methanol, ethanol, isopropanol, acetone, ethyl acetate, n-hexane, water and their mixtures; more preferably the solvent used for dissolution is isopropanol, water or their mixtures.

The present invention provides voriconazole, obtained by the process described herein, having a high enantiomeric purity of at least about 97% as measured by HPLC, preferably at least about 99% as measured by HPLC and more preferably at least about 99.8% as measured by HPLC; a chemical purity of at least about 98% as measured by HPLC, preferably at least about 99.5% as measured by HPLC, and more preferably at least about 99.8% as measured by HPLC; and substantially free of one or more of the following chiral impurities
i) (2S,3R)-2-(2,4-difluorophenyl)-3-(5-fluoropyrimidin-4-yl)-1-(1,2,4-triazol-1-yl)-2-butanol of formula IA.

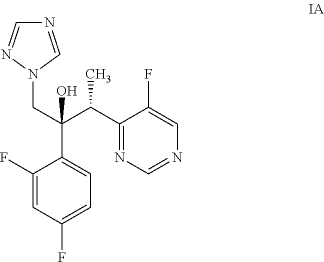

ii) (2S,3S)-2-(2,4-difluorophenyl)-3-(5-fluoropyrimidin-4-yl)-1-(1,2,4-triazol-1-yl)-2-butanol of formula IB.

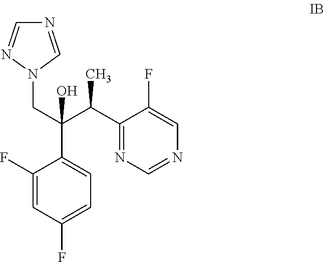

iii) (2R,3R)-2-(2,4-difluorophenyl)-3-(5-fluoropyrimidin-4-yl)-1-(1,2,4-triazol-1-yl)-2-butanol of formula IC.

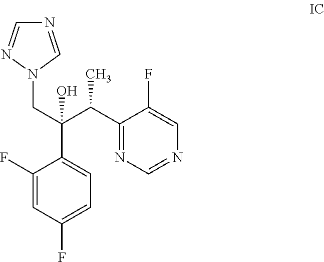

wherein the word “substantially free” refers to voriconazole having less than about 0.1%, of formula IA or IB or IC, as measured by HPLC, more preferably less than about 0.05% of formula IA or IB or IC, as measured by HPLC.

The present invention provides a voriconazole or a pharmaceutically acceptable salt thereof, obtained by the above process, as analyzed chemical purity using high performance liquid chromatography (“HPLC”) with the conditions described below:

### Column: C₁₈ Phenomenex Gemini, 250×4.6 mm, 5μ

### Column Temperature: 25° C.

Mobile phase: Mobile phase A=0.2% triethylamine in water. Adjust pH to 3.0 with o-phosphoric acid.
Mobile Phase B=Acetonitrile:Methanol (50:50, v/v)

| Time(min.) | % Mobile Phase A | Mobile Phase B |
|---|---|---|
| 0.01 | 60 | 40 |
| 30 | 55 | 45 |
| 45 | 20 | 80 |
| 50 | 20 | 80 |
| 55 | 60 | 40 |
| 60 | 60 | 40 |

Diluent: Mobile Phase A:Mobile Phase B (60:40, v/v)
Flow Rate: 1.5 mL/minute

### Detection: UV 254 nm

### Injection Volume: 20 mL

The present invention provides a voriconazole or a pharmaceutically acceptable salt thereof, obtained by the above process, as analyzed enantiomeric purity using high performance liquid chromatography (“HPLC”) with the conditions described below:

### Column: Chiralcel OD-H; 250×4.6 mm

### Column Temperature: 25° C.

Equlibration time: minimum 1.0 hour
Mobile Phase: n-Hexane:Ethanol:Diethylamine (80:20:0.2, v/v)

### Diluent: Mobile Phase

Flow Rate: 1.0 mL/minute

### Detection: UV 254 nm

### Injection Volume: 20 mL

Run time: 30 minutes

The present invention further provides voriconazole, obtained by the processes described herein, having relatively low content of one or more organic volatile impurities.

The present invention provides voriconazole, obtained using the processes described herein, may have a residual solvent content that is within the limits given by the International Conference on Harmonization of Technical Requirements for Registration of Pharmaceuticals for Human Use (ICH) guidelines. The guideline solvent level depends on the type of solvent but is not more than about 5000 ppm, or about 4000 ppm, or about 3000 ppm.

The present invention provides voriconazole, obtained by the process disclosed herein, having less than about 800 parts per million (ppm) C₁₋₄ alcohols such as methanol, ethanol, isopropanol, preferably less than about 200 ppm; less than about 500 ppm ethyl acetate, preferably less than about 100 ppm; less than about 500 ppm acetone, preferably less than about 100 ppm; less than about 500 ppm toluene, preferably less than about 100 ppm; less than about 500 ppm cyclohexane, preferably less than about 200 ppm; less than about 500 ppm tetrahydrofuran, preferably less than about 100 ppm; less than about 500 ppm acetic acid, preferably less than about 100 ppm; less than about 250 ppm petroleum ether, preferably less than about 100 ppm; less than about 500 ppm dichloromethane, preferably less than about 100 ppm; less than about 500 ppm n-hexane, preferably less than about 100 ppm; less than about 500 ppm n-heptane, preferably less than about 100 ppm; less than about 500 ppm dimethyl formamide, preferably less than about 100 ppm; less than about 200 ppm triethyl amine, preferably less than about 10 ppm.

As used herein, the term “residue” refers to a chemical state well known among pharmaceutical chemists wherein the recited pharmaceutical ingredient has not been separated as relatively semisolid, solid or powdery state from the medium, before it is mixed with other pharmaceutical ingredients.

The present invention further provides voriconazole, as disclosed herein for use in a pharmaceutical composition, previously described, which may independently have a D₅₀ and D₉₀ particle size less than about 300 microns, preferably less than about 200 microns, more preferably less than about 150 microns, still more preferably less than about 50 microns and most preferably less than about 10 microns. Whereupon, the notation Dₓ means that X% of particles have a diameter less than a specified diameter D. Thus, a D₅₀ of about 300 microns means that 50% of the micronized particles in a composition have a diameter less than about 300 microns. Any milling, grinding, micronizing or other particle size reduction method known in the art can be used to bring the solid state voriconazole into any desired particle size range set forth above.

The following examples are provided to enable one skilled in the art to practice the invention and are merely illustrative of the invention. The examples should not be read as limiting the scope of the invention as defined in the features and advantages.

### EXAMPLES

### Example 1

### The preparation of 4-chloro-6-ethyl-5-fluoropyrimidine of formula IV

100 gm of 6-ethyl-5-fluoro-4-hydroxy pyrimidine was charged under stiffing into a reactor containing 250 ml of methylene dichloride (MDC). 99 ml of triethylamine was added to the reaction mass and the reaction mass was cooled to about 5° C. to about 10° C. 73 ml of phosphorous oxychloride was added to the reaction mass at below about 10° C. in about 2 hours. The reaction mass was heated to about 45° C. to about 55° C. and stirred at reflux temperature for about 6 hours. The reaction mass was cooled to room temperature and was quenched into 438 ml of 3N HCl at below about 15° C. and stirred for about 30 minutes. MDC layer was separated and the aqueous layer was extracted with MDC. Combined the MDC layers and washed with water and dried over sodium sulphate and treated with 5 gm of charcoal and filtered through hyflo bed. Distilled-off MDC under vacuum below about 35° C. to obtain an oily mass. 25 ml of tetrahydrofuran (THF) is added to the oily mass and then distilled off THF under vacuum below about 45° C. to obtain 110 gm of pure 4-chloro-6-ethyl-5-fluoropyrimidine as an oily mass.

### Example 2

### The preparation of (2R,3S/2S,3R):(2R,3R/2S,3S)-3-(4-chloro-5-fluoropyrimidin-6-yl)-2-(2,4-difluorophenyl)-1-(1H-1,2,4-triazole-1-yl)butan-2-ol of Formula III

272 ml of diisopropylamine was charged into a reactor containing a mixture of 2.6 liters of n-heptane and 80 ml of tetrahydrofuran under a nitrogen atmosphere and cooled to about −20° C. to about −30° C. 779 ml of n-butyl lithium (1.6 molar solutions in n-hexane) was added to the reaction mass, while maintaining the temperature at about −20° C. to about −30° C. The reaction mixture was stirred for about 1 hr at the same temperature and then cooled the reaction mass to about −70° C. to about −80° C. 100 gm of 4-chloro-6-ethyl-5-fluoropyrimidine of formula IV in solution with 100 ml of tetrahydrofuran was added at about −70° C. to about −80° C. The reaction mixture was stirred for about 15 minutes at about the same temperature and then 278.9 gm of 1-(2,4-difluorophenyl)-2-(1H-1,2,4-triazol-1-yl)ethanone of formula V in solution with 1.5 liters of tetrahydrofuran was added at about −55° C. to about −80° C. The reaction mixture was stirred for about 2 hrs at about −70° C. to about −80° C. 140 ml of glacial acetic acid was added slowly to the reaction mass at about −60° C. to about −70° C. 3 liters of water was added slowly at below about −10° C. The reaction mixture was stirred for about 30 minutes at about 25° C. to about 30° C. and the solid filtered through a Buchner funnel. The organic layer was separated from the resultant filtrate and washed with water (3×1.5 liters). Concentrating the organic layer under vacuum (750 mm/Hg) at temperature below about 60° C. to yield 190 gms of the title compound as residue.

### Example 3

### The preparation of (2R,3S/2S, 3R)-2-(2,4-difluorophenyl)-3-(5-fluoropyrimidin-4-yl)-1-(1H-1,2,4-triazol-1-yl)-2-butanol (racemic voriconazole) of formula II

190 gm of (2R,3S/2S,3R):(2R,3R/2S,3S)-3-(4-chloro-5-fluoropyrimidin-6-yl)-2-(2,4-difluorophenyl)-1-(1H-1,2,4-triazole-1-yl)butan-2-ol of Formula III was charged into a autoclave reactor containing 1.71 liters of ethyl acetate and 190 ml of water. Charged 36.86 gm of sodium acetate and stirred for about 30 minutes at about 25° C. to about 30° C. 58.14 gm of sodium acetate and 3.0 gm of 10% Pd/C were charged and a 5 kg/cm² hydrogen pressure was applied. The reaction mixture was stirred for about 6 hrs to about 14 hrs at about 25° C. to about 30° C. under 5 kg/cm² hydrogen pressure. The catalyst was recovered by filtration through hyflo bed and washed with 100 ml of ethyl acetate. Charged 950 ml of water and 57 gm of sodium carbonate to the filtrate and stirred for about 30 minutes at about 20° C. to about 25° C. Separated the organic layer and extracted the aqueous layer with 380 ml of ethyl acetate. Combined the organic layer and washed with 760 ml of water. Charged 10 gm of charcoal to the organic layer at about 20° C. to about 25° C. and stirred for about 30 minutes. Filtered the charcoal through hyflo bed and washed with 95 ml of ethyl acetate. The filtrate was charged into the reactor and the solvent was removed by distillation under reduced pressure at below about 55° C. until a thick mass separated. Added 190 ml of isopropanol and stirred at about 55° C. to about 60° C. for about 30 minutes, then removed the isopropanol by distillation under reduced pressure at below 55° C. until a thick yellowish solid crude racemic voriconazole mass obtained (150 gm).

Enantiomeric purity by HPLC: 45%
Chemical purity by HPLC: 55%

### Example 4

### Purification of Racemic Voriconazole (Using Isopropanol+n-Hexane Solvent)

50 grams of the crude racemic voriconazole, obtained from example 3 was dissolved in 25 ml of isopropanol at about 65° C. to about 70° C. and stirred for about 30 minutes. Then 225 ml of n-hexane was added to the resultant solution at about 50° C. to about 55° C., and cooled the solution to about 25° C. to about 30° C. and stirred for about 12 hrs at about the same temperature. Again cooled to about 0° C. to about 10° C. and stirred for about 3 hrs at about the same temperature. Filtered the product and washed with 225 ml of n-hexane. The solid obtained was dried at about 55° C. to about 60° C. for about 12 hrs in air oven to yield 25 grams of pure racemic voriconazole.

Enantiomeric purity by HPLC: 45%
Chemical purity by HPLC: 90%
The XRD is set forth in FIG. 1.
The DSC is set forth in FIG. 2.

### Example 5

### Purification of Racemic Voriconazole (Using Isopropanol+n-Heptane Solvent)

50 grams of the crude racemic voriconazole, obtained from example 3 was dissolved in 25 ml of isopropanol at about 65° C. to about 70° C. and stirred for about 30 minutes. Then 225 ml of n-heptane was added to the resultant solution at about 50° C. to about 55° C., and cooled the solution to about 25° C. to about 30° C. and stirred for about 12 hrs at about the same temperature. Again cooled to about 0° C. to about 10° C. and stirred for about 3 hrs at about the same temperature. Filtered the product and washed with 225 ml of n-heptane. The solid obtained was dried at about 55° C. to about 60° C. for about 12 hrs in air oven to yield 26 grams of pure racemic voriconazole.

Enantiomeric purity by HPLC: 40%
Chemical purity by HPLC: 65%

### Example 6

### Preparation of Racemic Voriconazole Hydrochloride Salt

155 gm of (2R,3S/2S,3R):(2R,3R/2S,3S)-3-(4-chloro-5-fluoropyrimidin-6-yl)-2-(2,4-difluorophenyl)-1-(1H-1,2,4-triazole-1-yl)butan-2-ol of Formula III was charged into a autoclave reactor containing 1.1 liters of ethyl acetate and 100 ml of water. Charged 36.86 gm of sodium acetate and stirred for about 30 minutes at about 25° C. to about 30° C. 58.14 gm of sodium acetate and 2.85 gm of 10% Pd/C were charged and a 5 kg/cm² hydrogen pressure was applied. The reaction mixture was stirred for about 8 hrs to about 12 hrs at about 25° C. to about 30° C. under 5 kg/cm² hydrogen pressure. The catalyst was recovered by filtration through hyflo bed and washed with 100 ml of ethyl acetate. Charged 950 ml of water and 57 gm of sodium carbonate to the filtrate and stirred for about 30 minutes at about 20° C. to about 25° C. Separated the organic layer and extracted the aqueous layer with 380 ml of ethyl acetate. Combined the organic layer and washed with 760 ml of water. Charged 10 gm of charcoal to the organic layer at about 20° C. to about 25° C. and stirred for about 30 minutes. Filtered the charcoal through hyflo bed and washed with 95 ml of ethyl acetate. The filtrate was charged into the reactor and the solvent was removed by distillation under reduced pressure at about below 55° C. until a thick oily mass separated. This oily mass was dissolved in 775 ml of ethylacetate under stirring, cooled to about 10° C. to about 15° C. 65 ml of 18% isopropanolic hydrochloride solution added slowly under stirring. Maintained under stirring for about 12 hrs at about 20° C. to about 25° C. and cooled to about 0° C. to about 5° C. and filtered the resultant precipitate through Buchner funnel and washed with 200 ml of n-hexane. The wet compound was dried in air oven at about 50° C. to about 55° C. to get about 60 gm of racemic voriconazole hydrochloride salt.

HPLC purity: 95%

### Example 7

### Preparation of Racemic Voriconazole (from Racemic Voriconazole Hydrochloride Salt)

60 gms of racemic voriconazole hydrochloride salt, obtained from example 6 was added to 1.5 liters of MDC. Saturated potassium carbonate solution was added (30 gm of solid potassium carbonate dissolved in 300 ml of water) until pH was above 10 (pH: 8-10) and continued stiffing for 30 minutes. MDC layer was separated and the aqueous layer was extracted with MDC. Combined MDC layers and evaporated under vacuum to get crude racemic voriconazole. Added 25 ml of isopropanol at about 65° C. to about 70° C. and stirred for about 30 minutes. Then 225 ml of n-hexane was added to the resultant solution at about 50° C. to about 55° C., and cooled the solution to about 25° C. to about 30° C. and stirred for about 12 hrs at the same temperature. Again cooled to about 0° C. to about 10° C. and stirred for about 3 hrs at the same temperature. Filtered the product and washed with 225 ml of n-hexane. The solid obtained was dried at about 55° C. to about 60° C. for about 12 hrs in air oven to yield 24 grams of pure racemic voriconazole.

### Example 8

### Preparation of Racemic Voriconazole (±)-Camphorsulphonate Salt

155 gm of (2R,3S/2S,3R):(2R,3R/2S,3S)-3-(4-chloro-5-fluoropyrimidin-6-yl)-2-(2,4-difluorophenyl)-1-(1H-1,2,4-triazole-1-yl)butan-2-ol of Formula III was charged into a autoclave reactor containing 1.1 liters of ethyl acetate and 100 ml of water. Charged 36.86 gm of sodium acetate and stirred for about 30 minutes at about 25° C. to about 30° C. 58.14 gm of sodium acetate and 2.85 gm of 10% Pd/C were charged and a 5 kg/cm² hydrogen pressure was applied. The reaction mixture was stirred for about 8 hrs to about 12 hrs at about 25° C. to about 30° C. under 5 kg/cm² hydrogen pressure. The catalyst was recovered by filtration through hyflo bed and washed with 100 ml of ethyl acetate. Charged 950 ml of water and 57 gm of sodium carbonate to the filtrate and stirred for about 30 minutes at about 20° C. to about 25° C. Separated the organic layer and extracted the aqueous layer with 380 ml of ethyl acetate. Combined the organic layer and washed with 760 ml of water. Charged 10 gm of charcoal to the organic layer at about 20° C. to about 25° C. and stirred for about 30 minutes. Filtered the charcoal through hyflo bed and washed with 95 ml of ethyl acetate. The filtrate was charged into the reactor and the solvent was removed by distillation under reduced pressure at about below 55° C. until a thick oily mass separated. The resultant thick oily mass was dissolved in 870 ml of acetone and 290 ml of methanol. Added 96.28 gm of R-(−)-10-Camphore-10-sulphonic acid & 96.28 gm of the S-(+)-10-Camphor-10-sulphonic acid and stirred for about 30 minutes at about 25° C. to about 30° C. Maintained the reaction mass for 12 hours at same temperature and further cooled to about 0° to about 5° C. Filtered the precipitated compound through Buchner funnel and washed with 145 ml of Acetone. The solid obtained was dried at about 55° C. to about 60° C. for about 12 hrs in air oven to yield 130 gm of racemic voriconazole racemic camphorsulphonate salt as a solid.

HPLC purity: 95%

### Example 9

### Preparation of Racemic Voriconazole (from Racemic Voriconazole (±)-Camphorsulphonate Salt)

130 gm of racemic voriconazole (±)-camphorsulphonate salt, obtained from example 8 was added to 410 ml of MDC and 410 ml of purified water and stirred for about 30 minutes. 31.2 ml of 40% sodium hydroxide solution (40 gm of sodium hydroxide dissolved in 100 ml of water) was added slowly to the reaction mass until pH was above 10 (pH: 9-12) and stirred for about 30 minutes. Aqueous and organic layers were separated and the aqueous layer was extracted with MDC. Combined MDC layers and washed with purified water. Separated the MDC layer and treated with 5 gm of charcoal and filtered through hyflo bed. MDC layer was evaporated under vacuum below about 45° C. to get crude racemic voriconazole. Added 260 ml of isopropanol at about 65° C. to about 70° C. and stirred for about 30 minutes. Cooled the solution to about 25° C. to about 30° C. and stirred for about 12 hrs at the same temperature. Again cooled to about 0° C. to about 10° C. and stirred for about 3 hrs at about the same temperature. Filtered the product and washed with 130 ml of isopropanol. The solid obtained was dried at about 55° C. to about 60° C. for about 12 hrs in air oven to yield 65 grams of pure racemic voriconazole.

HPLC purity: 98%

### Example 10

### Preparation of (1R)-(−)-10-camphorsulfonate salt of (2R,3S)-2-(2,4-difluorophenyl)-3-(5-fluoropyrimidin-4-yl)-1-(1H-1,2,4-triazol-1-yl)-2-butanol (voriconazole (1R)-(−)-10-camphorsulfonate salt)

65 gm of (2R,3S/2S,3R)-2-(2,4-difluorophenyl)-3-(5-fluoropyrimidin-4-yl)-1-(1H-1,2,4-triazol-1-yl)-2-butanol (racemic voriconazole) of formula II, obtained from example 4 was charged into a reactor containing 520 ml of acetone and 130 ml of methanol. 20.73 gm of R(−)-10-camphor sulfonic acid was added to the reaction mass to make a clear solution. The reaction solution was stirred for about 15 hrs at about 20° C. to about 25° C. Cooled the solution gradually to about 0° C. to about −5° C. for about 4 hrs. Filtered the product and washed with 65 ml of chilled acetone. The obtained solid was dried at about 45° C. to about 50° C. for about 12 hrs to yield 31 gms of the title compound.

### Yield: 47.69%

Enantiomeric purity by HPLC: 99.51%
Chemical purity by HPLC: 99.2%

### Example 11

### The preparation of (2R,3S)-2-(2,4-difluorophenyl)-3-(5-fluoropyrimidin-4-yl)-1-(1H-1,2,4-triazol-1-yl)-2-butanol (voriconazole) of formula I

30 gm of (1R)-(−)-10-camphorsulfonate salt of (2R,3S)-2-(2,4-difluorophenyl)-3-(5-fluoropyrimidin-4-yl)-1-(1H-1,2,4-triazol-1-yl)-2-butanol (voriconazole (1R)-(−)-10-camphorsulfonate salt), obtained from Example 10 was charged into a reactor containing 97.5 ml of dichloromethane and 97.5 ml of water. Reaction mass pH was adjusted to about 11 to about 14 with 6 ml of 40% aqueous sodium hydroxide solution. The reaction mass was stirred for about 15 minutes. The organic layer was separated and aqueous layer was extracted with dichloromethane (3×30 ml). All the organic layers were combined and washed with water (3×70 ml). Separated the organic layer and charged 1.5 gm of charcoal. Stirred for about 15 minutes at about 25° C. to about 30° C. Filtered the charcoal through hyflo bed and washed with dichloromethane (2×20 ml). The filtrate was charged into the reactor and the solvent was removed by distillation under atmospheric pressure at below 45° C. Added 15 ml of isopropanol and removed by distillation under reduced pressure at a temperature below 55° C. until a thick mass was obtained. Charged 60 ml of isopropanol to the resultant residue and stirred for about 30 minutes at about 60° C. to about 70° C. Cooled the reaction mass to about 20° C. to about 25° C. and then to about 0° C. about 5° C. and stirred for about 2 hrs at the same temperature. Filtered the product and washed with 20 ml of chilled isopropanol. The solid obtained was dried at about 50° C. to about 55° C. for about 12 hrs in an air oven to yield 14.5 gms of the title compound.

| | |
|---|---|
| 1) | Enantiomeric purity by HPLC: 99.99% |
| 2) | Formula IA: 0.01% |
| 3) | Formula IB: 0.01% |
| 4) | Formula IC: 0.02% |
| 5) | Chemical purity by HPLC: 99.98% |

1. A process for preparation of 2-(2,4-difluorophenyl)-3-(5-fluoropyrimidin-4-yl)-1-(1-1H-1,2,4-triazol-1-yl)-2-butanol (racemic voriconazole) of formula II or a pharmaceutically acceptable salt thereof in a single reaction vessel, comprising; 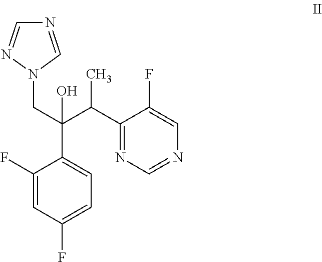
a) condensing 4-chloro-6-ethyl-5-fluoro pyrimidine of formula IV with 1-(2,4-difluorophenyl)-2-(1H-1,2,4-triazole-1-yl)ethanone of formula V in presence of an organic metallic base and an organic solvent S1, 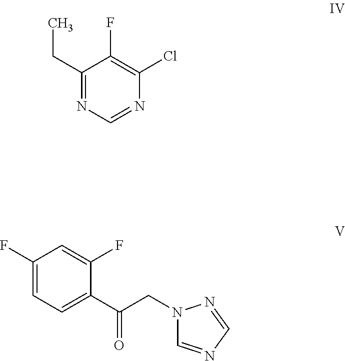
b) concentrating the reaction mixture to obtain 3-(4-chloro-5-fluoropyrimidin-6-yl)-2-(2,4-difluorophenyl)-1-(1H-1,2,4-triazole-1-yl)butan-2-ol of formula III as residue, 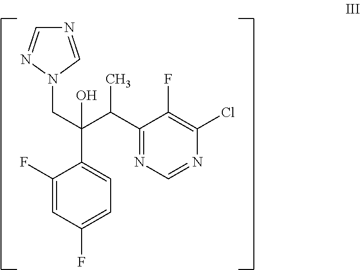
c) dechlorination of the resulting compound of formula III as a residue under catalytic hydrogenation by using a metal catalyst in an organic solvent S2, and
d) isolating the racemic voriconazole of formula II or a pharmaceutically acceptable salt thereof.

2. (canceled)

3. The process of claim 1, wherein the organic metallic base is selected from lithium diisopropylamide, magnesium isopropylamide, sodium bis(trimethylsilyl)amide, potassium bis(trimethylsilyl)amide, butyl magnesium, zinc isopropyl amide, butyl zinc, lithium hexamethyl disilazane, sodium hexamethyl disilazane, magnesium hexamethyl disilazane.

4. The process of claim 1, wherein the organic solvent S1 is selected from ether, tetrahydrofuran; hydrocarbons selected from hexane, heptane, cyclohexane; and mixtures thereof.

5. The process of claim 4, wherein the organic metallic base is lithium diisopropylamide and the organic solvent S1 is mixture of tetrahydrofuran and hexane.

6. The process of claim 1, concentrating the reaction mixture by means of evaporation under atmospheric pressure or evaporation under vacuum.

7. The process of claim 1, wherein the metal catalyst is selected from palladium catalyst, or Raney nickel.

8. The process of claim 1, wherein the organic solvent S2 is selected from C₁-C₄ alcohols, C₁-C₅ esters, water and mixtures thereof.

9. The process of claim 8, wherein the organic solvent S2 is methanol, ethanol, ethyl acetate, water and mixtures thereof.

10. The process of claim 1, wherein the metal catalyst is palladium catalyst and the organic solvent S2 is mixture of ethyl acetate and water.

11. A process for preparation of 2-(2,4-difluorophenyl)-3-(5-fluoropyrimidin-4-yl)-1-(1-1H-1,2,4-triazol-1-yl)-2-butanol (racemic voriconazole) of formula II or a pharmaceutically acceptable salt thereof, comprising;
a. forming an intermediate compound 3-(4-chloro-5-fluoropyrimidin-6-yl)-2-(2,4-difluorophenyl)-1-(1H-1,2,4-triazole-1-yl)butan-2-ol of formula III
by condensing 4-chloro-6-ethyl-5-fluoro pyrimidine of formula IV with 1-(2,4-difluorophenyl)-2-(1H-1,2,4-triazole-1-yl)ethanone of formula V in presence of an organic metallic base and an organic solvent S1
b. concentrating the reaction mixture to obtain 3-(4-chloro-5-fluoropyrimidin-6-yl)-2-(2,4-difluorophenyl)-1-(1H-1,2,4-triazole-1-yl)butan-2-ol of formula III as residue
c. dechlorination of the resulting compound of formula III as a residue under catalytic hydrogenation by using a metal catalyst in an organic solvent S2 to yield racemic voriconazole of formula II, wherein the compound of formula III is not crystallized out before dechlorination
d. isolating the racemic voriconazole of formula II or a pharmaceutically acceptable salt thereof.

12. The process as in claim 1, wherein the racemic voriconazole is isolated as hydrochloride salt.

13. The process as in claim 1, wherein the racemic voriconazole is isolated as racemic camphor sulfonic acid salt.

14. A process for the purification of racemic voriconazole, comprising;
a) dissolving racemic voriconazole obtained from the process of claim 1, in a suitable organic solvent, wherein the suitable organic solvent is selected from methanol, ethanol, isopropanol, butanol and mixtures thereof,
b) heating the mixture of a) at a temperature sufficient to obtain a solution,
c) adding an antisolvent to the resultant solution in b) to precipitate the racemic voriconazole or a pharmaceutically acceptable salt thereof, wherein the antisolvent is a hydrocarbon solvent selected from n-hexane, n-heptane, pentane, cyclohexane and toluene and mixtures thereof,
d) cooling the precipitate and recovering the precipitate.

15. The process of claim 14, wherein the suitable organic solvent is isopropanol, and the anti solvent is n-hexane or n-heptane.

16. The process of claim 14, wherein the temperature sufficient to obtain a solution is about 40° C. to about 80° C.

17. (canceled)

18. The process of claim 14, wherein the cooling the precipitate is carried out at a temperature of about 0° C. to about 30° C.

19. The process of claim 14, wherein the resultant racemic voriconazole is crystalline Form A.

20.-21. (canceled)

22. A process or preparation of (2R,3S)-2-(2,4-difluorophenyl)-3-(5-fluoropyrimidin-4-yl)-1-(1,2,4-triazol-1-yl)-2-butanol (Voriconazole) of formula I or a pharmaceutically acceptable salt thereof, comprising; 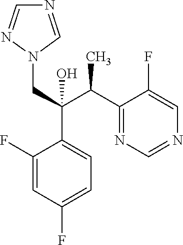
a) reacting the 2-(2,4-difluorophenyl)-3-(5-fluoropyrimidin-4-yl)-1-(1,2,4-triazol-1-yl)-2-butanol of formula II obtained from the process of claim 1, with (1R)-(−)-10-camphorsulfonic acid,
b) neutralizing the resulting (2R,3S)-2-(2,4-difluorophenyl)-3-(5-fluoropyrimidin-4-yl)-1-(1,2,4-triazol-1-yl)-2-butanol (1R)-(−)-10-camphorsulfonate salt.

23.-26. (canceled)

27. A pharmaceutical composition comprising voriconazole obtained from the process of claim 22, together with one or more pharmaceutically acceptable excipients.
